SEQUENCE LISTING (67 sequences)

SEQ ID NO: 1 - protein, 12 aa, Artificial
  Peptide (TAT) derived from protein transduction domain sequence of HIV-1
GYGRKKRRQRRR

SEQ ID NO: 2 - protein, 16 aa, Artificial
  Peptide (AntP) derived from protein transduction domain of Droshophila antennapedia gene homeodomaain
RQIKIWFQNRRMKWKK

SEQ ID NO: 3 - protein, 16 aa, Artificial
  Peptide (PER1-1) derived from protein transduction domain of human period-1 protein
SRRHHCRSKAKRSRHH

SEQ ID NO: 4 - protein, 14 aa, Artificial
  Peptide (PER1-2) derived from protein transduction domain of human period-1 protein
GRRHHRRSKAKRSR

SEQ ID NO: 5 - protein, 9 aa, Artificial
  Peptide (gp100-280-288 (9v)) derived from a melanocyte differentiation antigen, gp100
YLEPGPVTV

SEQ ID NO: 6 - protein, 9 aa, Artificial
  Peptide (gp100-154-162) derived from a melanocyte differentiation antigen, gp100
KTWGQYWQV

SEQ ID NO: 7 - protein, 9 aa, Artificial
  Peptide (MART-1 32) derived from a melanocyte differentiation antigen
ILTVILGVL

SEQ ID NO: 8 - protein, 9 aa, Artificial
  Peptide (MART-1 31) derived from a melanocyte differentiation antigen
GILTVILGV

SEQ ID NO: 9 - protein, 9 aa, Artificial
  Peptide (MART-1 99) derived from a melanocyte differentiation antigen
NAPPAYEKL

SEQ ID NO: 10 - protein, 9 aa, Artificial
  Peptide (MART-1 1) derived from a melanocyte differentiation antigen
MPREDAHFI

SEQ ID NO: 11 - protein, 9 aa, Artificial
  Peptide (MART-1 56) derived from a melanocyte differentiation antigen
ALMDKSLHV

SEQ ID NO: 12 - protein, 9 aa, Artificial
  Peptide (MART-1 39) derived from a melanocytle differentiation antigen
VLLLIGCWY

SEQ ID NO: 13 - protein, 9 aa, Artificial
  Peptide (MART-1 35) derived from a melanocyte differentiation antigen
VILGVLLLI

SEQ ID NO: 14 - protein, 9 aa, Artificial
  Peptide (MART-1 61) derived from a melanocyte differentiation antigen
SLHVGTQCA

SEQ ID NO: 15 - protein, 9 aa, Artificial
  Peptide (MART-1 57) derived from melanocyte differentiation antigen
LMDKSLHVG

SEQ ID NO: 16 - protein, 9 aa, Artificial
  Peptide (MAGE-A3 115) derived from cancer- testis antigen
ELVHFLLLK

SEQ ID NO: 17 - protein, 9 aa, Artificial
  Peptide (MAGE-A3 285) derived from cancer -testis antigen
KVLHHMVKI

SEQ ID NO: 18 - protein, 9 aa, Artificial
  Peptide (MAGE-A3 276) derived from cancer- testis antigen
RALVETSYV

SEQ ID NO: 19 - protein, 9 aa, Artificial
  Peptide (MAGE-A3 105) derived from cancer- testis antigen
FQAALSRKV

SEQ ID NO: 20 - protein, 9 aa, Artificial
  Peptide (MAGE-A3 296) derived from cancer- testis antigen
GPHISYPPL

SEQ ID NO: 21 - protein, 9 aa, Artificial
  Peptide (MAGE-A3 243) derived from cancer- testis antigen
KKLLTQHFV

SEQ ID NO: 22 - protein, 9 aa, Artificial
  Peptide (MAGE-A3 24) derived from cancer- testis antigen
GLVGAQAPA

SEQ ID NO: 23 - protein, 9 aa, Artificial
  Peptide (MAGE-A3 301) derived from cancer- testis antigen
YPPLHEWVL

SEQ ID NO: 24 - protein, 9 aa, Artificial
  Peptide (MAGE-A3 71) derived from cancer- testis antigen
LPTTMNYPL

SEQ ID NO: 25 - protein, 9 aa, Artificial
  Peptide (Tyr 171) derived from a melanocyte differentiation antigen
NIYDLFVWM

SEQ ID NO: 26 - protein, 9 aa, Artificial
  Peptide (Tyr 444) derived from a melanocyte differentiation antigen
DLGYDYSYL

SEQ ID NO: 27 - protein, 9 aa, Artificial
  Peptide (Tyr 57) derived from melanocyte differentiation antigen
NILLSNAPL

SEQ ID NO: 28 - protein, 9 aa, Artificial
  Peptide (TRP-1 245) derived from a tumor associated antigen
SLPYWNFAT

SEQ ID NO: 29 - protein, 9 aa, Artificial
  Peptide (TRP-1 298) derived from tumor associated antigen
TLGTLCNST

SEQ ID NO: 30 - protein, 9 aa, Artificial
  Peptide (TRP-1 481) derived from tumor associated antigen
IAVVGALLL

SEQ ID NO: 31 - protein, 9 aa, Artificial
  Peptide (TRP-1 181) derived from tumor associated antigen
NISIYNYFV

SEQ ID NO: 32 - protein, 9 aa, Artificial
  Peptide (TRP-1 439) derived from tumor associated antigen
NMVPFWPPV

SEQ ID NO: 33 - DNA, 36 bp, Artificial
  Sequence encoding TAT
ggctacggcaggaagaagaggaggcagaggaggagg

SEQ ID NO: 34 - DNA, 48 bp, Artificial
  Sequence encoding AntP
aggcagatcaagatctggttccagaacaggaggatgaagtggaagaag

SEQ ID NO: 35 - DNA, 48 bp, Artificial
  Sequence encoding PER1-1
agcaggaggcaccactgcaggagcaaggccaagaggagcaggcaccac

SEQ ID NO: 36 - DNA, 42 bp, Artificial
  Sequence encoding PER1-2
ggcaggaggcaccacaggaggagcaaggccaagaggagcagg

SEQ ID NO: 37 - DNA, 27 bp, Artificial
  Sequence encoding gp100-280-288 (9V)
tacctggagcccggccccgtgaccgtg

SEQ ID NO: 38 - DNA, 27 bp, Artificial
  Sequence encoding gp100-154-162
aagacctggggccagtactggcaggtg

SEQ ID NO: 39 - DNA, 27 bp, Artificial
  Sequence encoding MART-1 32
atcctgacagtgatcctgggagtctta

SEQ ID NO: 40 - DNA, 27 bp, Artificial
  Sequence encoding MART-1 31
ggcatcctgacagtgatcctgggagtc

SEQ ID NO: 41 - DNA, 27 bp, Artificial
  Sequence encoding MART-1 99
aatgctccacctgcttatgagaaactc

SEQ ID NO: 42 - DNA, 27 bp, Artificial
  Sequence encoding MART-1 1
atgccaagagaagatgctcacttcatc

SEQ ID NO: 43 - DNA, 27 bp, Artificial
  Sequence encoding MART-1 56
gccttgatggataaaagtcttcatgtt

SEQ ID NO: 44 - DNA, 27 bp, Artificial
  Sequence encoding MART-1 39
gtcttactgctcatcggctgttggtat

SEQ ID NO: 45 - DNA, 27 bp, Artificial
  Sequence encoding MART-1 35
gtgatcctgggagtcttactgctcatc

SEQ ID NO: 46 - DNA, 27 bp, Artificial
  Sequence encoding MART-1 61
agtcttcatgttggcactcaatgtgcc

SEQ ID NO: 47 - DNA, 27 bp, Artificial
  Sequence encoding MART-1 57
ttgatggataaaagtcttcatgttggc

SEQ ID NO: 48 - DNA, 27 bp, Artificial
  Sequence encoding MAGE-A3 115
gagttggttcattttctgctcctcaag

SEQ ID NO: 49 - DNA, 27 bp, Artificial
  Sequence encoding MAGE-A3 285
aaagtcctgcaccatatggtaaagatc

SEQ ID NO: 50 - DNA, 27 bp, Artificial
  Sequence encoding MAGE-A3 276
agggccctcgttgaaaccagctatgtg

SEQ ID NO: 51 - DNA, 27 bp, Artificial
  Sequence encoding MAGE-A3 105
ttccaagcagcactcagtaggaaggtg

SEQ ID NO: 52 - DNA, 27 bp, Artificial
  Sequence encoding MAGE-A3 296
ggacctcacatttcctacccacccctg

SEQ ID NO: 53 - DNA, 27 bp, Artificial
  Sequence encoding MAGE-A3 243
aagaagctgctcacccaacatttcgtg

SEQ ID NO: 54 - DNA, 27 bp, Artificial
  Sequence encoding MAGE-A3 24
ggcctggtgggtgcgcaggctcctgct

SEQ ID NO: 55 - DNA, 27 bp, Artificial
  Sequence encoding MAGE-A3 301
tacccacccctgcatgagtgggttttg

SEQ ID NO: 56 - DNA, 27 bp, Artificial
  Sequence encoding MAGE-A3 71
ctccccactaccatgaactaccctctc

SEQ ID NO: 57 - DNA, 27 bp, Artificial
  Sequence encoding TYR 171
aatatttatgacctctttgtctggatg

SEQ ID NO: 58 - DNA, 27 bp, Artificial
  Sequence encoding TYR 444
gatctgggctatgactatagctatcta

SEQ ID NO: 59 - DNA, 27 bp, Artificial
  Sequence encoding TYR 57
aatatccttctgtccaatgcaccactt

SEQ ID NO: 60 - DNA, 27 bp, Artificial
  Sequence encoding TRP-1 245
tcccttccttactggaattttgcaacg

SEQ ID NO: 61 - DNA, 27 bp, Artificial
  Sequence encoding TRP-1 298
accctgggaacactttgtaacagcacc

SEQ ID NO: 62 - DNA, 27 bp, Artificial
  Sequence encoding TRP-1 481
atagcagtagttggcgctttgttactg

SEQ ID NO: 63 - DNA, 27 bp, Artificial
  Sequence encoding TRP-1 181
aacatttccatttataactactttgtt

SEQ ID NO: 64 - DNA, 27 bp, Artificial
  Sequence encoding TRP-1 439
aacatggtgccattctggcccccagtc

SEQ ID NO: 65 - DNA, 75 bp, Artificial
  Sequence encoding PER1-1 fused to gp100-280- 288 (9V)
agcaggaggcaccactgcaggagcaaggccaagaggagcaggcaccactacctggagcccggccccgtgaccgtg

SEQ ID NO: 66 - DNA, 96 bp, Artificial
  Sequence encoding PER1-2 fused to gp100 (154- 162)
ggcaggaggcaccacaggaggagcaaggccaagaggagcagggccagcaacgagaacatggagaccatgaagacctggggccagtactggcaggtg

SEQ ID NO: 67 - DNA, 111 bp, Artificial
  Sequence encoding PER1-2 fused to linker fused to gp100-154-162
ggcaggaggcaccacaggaggagcaaggccaagaggagcagggccagcaacgagaacatggagaccatgttcgtgtacgtgtggaagacctggggccagtactggcaggtg

(19) United States
(12) Patent Application Publication
(10) Pub. No.: US 2004/0002455 A1
(43) Pub. Date: Jan. 1, 2004

(54) Targeted immunogens

(75) Inventors: Robert Adam Uger, Richmond Hill, CA (US); Danielle Salha, Toronto (CA); Brian Barber, White Plains, NY (US); Clarence C. Morse, Asbury, NJ (US); Yong Guo, Freshmeadows, NJ (US); Su Cheng, Bridgewater, NJ (US)

Correspondence Address: Patrick J. Halloran, Aventis Pasteur, Inc., Intellectual Property, Knerr Building, One Discovery Drive, Swiftwater, PA, 18370, US

(73) Assignees: Aventis Pasteur, Ltd., Toronto (CA); Aventis Pharmaceuticals, Inc., Bridgewater, NJ

(21) Appl. No.: 10/353,678

(22) Filed: Jan. 29, 2003

Related U.S. Application Data
(63) Continuation-in-part of application No. 10/219,850, filed on Aug. 15, 2002.
(60) Provisional application No. 60/352,892, filed on Jan. 29, 2002.

(51) Int. Cl. A61K 38/17; C12P 21/02; C12N 5/06; C07K 14/705; C07H 21/04

(52) U.S. Cl. 514/12; 530/350; 435/69.1; 435/320.1; 435/325; 536/23.2

(57) ABSTRACT

The present invention provides reagents and methods for producing and utilizing targeted immunogens. In preferred embodiments, an immunogen is conjugated to an amino acid sequence that targets the immunogen to the MHC presentation pathway. Using the reagents and methods provided herein, immunization protocols may be enhanced resulting in increased immunity of the host.

### RELATED APPLICATIONS

This application claims priority U.S. Ser. No. 60/352,892 filed Jan. 29, 2002 and a continuation-in-part of U.S. Ser. No. 10/219,850 filed Aug. 15, 2002.

### FIELD OF THE INVENTION

The present invention relates to reagents and methods for improving immunization protocols. For instance, amino acid sequences that direct immunogenic amino acid sequences to the MHC presentation pathway.

### BACKGROUND OF THE INVENTION

Although peptide-based vaccines have a number of advantages (safety, ease of manufacture) they often exhibit limited immunogenicity. This is due, in part, to the inability of exogenous peptides to efficiently access the class I MHC presentation pathway. Thus, strategies that can enhance the delivery of peptides to MHC have the potential to increase the efficacy of peptide-based vaccines. One strategy is to link immunogenic sequences to “protein transduction domains” (PTD), which have been shown to drive translocation of proteins and peptides across cell membranes. Exemplary PTDs include HIV-Tat, cell penetrating peptides (CPP), Trojan carriers, Antennapedia homeodomain, and human period-1 protein.

In one approach, antigenic peptides are attached to a short cationic peptide derived from HIV-1 tat (i.e., residues 49-57) to form fusion conjugates. It has been shown that exposure of antigen presenting cells (“APC”), such as dendritic cells, process ova-tat conjugates resulting in stimulation of antigen-specific CD8⁺ T cells (Kim, et al. J Immunol Aug. 15, 1997;159(4):1666-8; Shibagaki, et al. J Immunol 2002 Mar 1 ;168(5):2393-401). This has also been demonstrated for the human melanoma antigen TRP2 (Wang, et al. J Clin Invest 2002 June;109(11):1463-70). Evidence to the contrary has been demonstrated following conjugation of the tat peptide to full-length proteins (Leifert, et al. Gene Ther 2002 November;9(21):1422-8).

In another approach, the Antennapedia homeodomain (AntpHD) has been fused to CTL epitopes and shown to enhance CD8⁺ T cell reactivity (Chikh, et al. J Immunol Dec. 1, 2001;167(11):6462-70; Pietersz, et al. Vaccine Jan. 8, 2001;19(11-12):1397-405; Schutze-Redelmeier, et al. J Immunol Jul. 15, 1996;157(2):650-5). AntpHD has been shown to be useful with antigenic sequences of up to 50 amino acids.

In other studies, the transduction sequence from the human period-i protein (hPER1, sequence SRRHHCRSKAKRSRHH) has been shown to efficiently cross cell membranes. It is therefore an attractive antigen delivery vehicle candidate. As shown below in detail, hPER1 does in fact operate to enhance antigen presentation and T cell reactivity.

### BRIEF DESCRIPTION OF THE DRAWINGS

FIG. 1. In vitro sensitization of target cells for peptide-specific lysis by hPER1 conjugates.

FIG. 2. In vitro induction of human T cell responses using a hPER1 conjugate peptide.

FIG. 3. In vivo induction of T cell responses using hPER1 conjugate peptides without adjuvant.

FIG. 4. In vitro analysis of OVA peptide presentation. Splenocytes from C57BL/6 mice were pulsed with 10 ug/ml of the indicated peptides for 1 hour at 37° C., washed, and incubated for 0, 4, 8, 24, or 30 hours. Cells pulsed with transduction peptides were pre-incubated with a bGAL peptide to block any cell surface binding. The cells were then tested by ELISPOT for their ability to induce IFN-γ secretion from SIINFEKL-specific T cells. Spot counts greater than 300/well could not be counted. *=sample not tested.

FIG. 5. In vitro analysis of NP peptide presentation. Splenocytes from C57BL/6 mice were pulsed with 10 ug/ml of the indicated peptides for 1 hour at 37C, washed, and incubated for 0, 24, 72, or 120 hours. Cells were then tested by ELISPOT for their ability to induce IFN-γ secretion from NP-specific T cells.

FIG. 6. FIG. 3: CTL responses in C57BL/6 mice following i.v. injection of peptide-pulsed DCs. Mice were immunized iv with 5×10e5 bone marrow-derived DCs pulsed with the indicated peptides. Splenocytes from vaccinated animals were harvested one week post immunization, restimulated with the native OVA peptide for 5 days, and tested for CTL activity in a standard chromium release assay using target cells pulsed with OVA peptide.

FIG. 7. CTL responses in HLA-A2/Kb transgenic mice following s.c. injection of peptide. Mice were immunized s.c. with 50 ug of the indicated peptides and boosted on days 21 and 42 following the first injection. Splenocytes from immunized animals were harvested on day 63 post immunization, restimulated with the native gp100-154 peptide for 5 days, and tested for CTL activity in a standard chromium release assay using target cells pulsed with gp100-154 peptide.

### SUMMARY OF THE INVENTION

The present invention provides reagents and methods for producing and utilizing targeted immunogens. In preferred embodiments, an immunogen is conjugated to an amino acid sequence that targets the immunogen to the MHC presentation pathway. Using the reagents and methods provided herein, immunization protocols may be enhanced resulting in increased immunity of the host.

### DETAILED DESCRIPTION

The present invention provides methods for targeting immunogens to Class I MHC using amino acid sequences the preferentially direct a peptide to the MHC presentation pathway (referred to herein as a “targeting sequence”). This targeting strategy may be utilized in peptide-based immunization protocols, for expression of antigens in dendritic cells, in nucleic acid vaccines, and viral vector vaccination, for example. For the purposes of describing the present invention, an immunogenic amino acid sequence linked to a targeting amino acid sequence is referred to as a “targeted immunogen”. The term “targeted immunogen” includes fragments, variants, or derivatives thereof.

The targeting sequences may include, for example, a transduction sequence of Antennapedia, TAT, VP22, or hPER1 (i.e., targeting sequences). Preferred targeting sequences include, for example:

| | | |
|---|---|---|
| TAT: | GYGRKKRRQRRR | (SEQ ID NO.:1) |
| AntP: | RQIKIWFQNRRMKWKK | (SEQ ID NO.:2) |
| PER1-1: | SRRHHCRSKAKRSRHH | (SEQ ID NO.:3) |
| PER1-2: | GRRHHRRSKAKRSR | (SEQ ID NO.:4) |

In one embodiment, cytotoxic T lymphocyte (CTL) epitopes are joined to the hPER1 transduction sequence to form targeted immunogens (or “hPER1-CTL conjugates”). It is preferred that administration of a targeted immunogen to a host results in an anti-immunogen immune response that is greater than that obtained using the immunogen alone (i.e., increased cytotoxic T cell response).

Suitable immunogens may also include, for example, peptide sequences of tumor antigens (TA). The term “TA” includes both tumor-associated antigens (TAAs) and tumor-specific antigens (TSAs), where a cancerous cell is the source of the antigen. A TAA is an antigen that is expressed on the surface of a tumor cell in higher amounts than is observed on normal cells or an antigen that is expressed on normal cells during fetal development. A TSA is an antigen that is unique to tumor cells and is not expressed on normal cells. TA further includes TAAs or TSAs, antigenic or immunogenic fragments thereof, and modified versions that retain their antigenicity and/or immunogenecity. TAs are typically classified into five categories according to their expression pattern, function, or genetic origin: cancer-testis (CT) antigens (i.e., MAGE, NY-ESO-1); melanocyte differentiation antigens (i.e., Melan A/MART-1, tyrosinase, gp100); mutational antigens (i.e., MUM-i, p53, CDK-4); overexpressed ‘self’ antigens (i.e., HER-2/neu, p53); and, viral antigens (i.e., HPV, EBV). Suitable TAs include, for example, gp100 (Cox et al., Science, 264:716-719 (1994)), MART-1/Melan A (Kawakami et al., J. Exp. Med., 180:347-352 (1994)), gp75 (TRP-1) (Wang et al., J. Exp. Med., 186:1131-1140 (1996)), tyrosinase (Wolfel et al., Eur. J. Immunol., 24:759-764 (1994)), NY-ESO-1 (WO 98/14464; WO 99/18206), melanoma proteoglycan (Hellstrom et al., J. Immunol., 130:1467-1472 (1983)), MAGE family antigens (i.e., MAGE-1,2,3,4,6, and 12; Van-der Bruggen et al., Science, 254:1643-1647 (1991); U.S. Pat. Nos. 6,235,525), BAGE family antigens (Boel et al., Immunity, 2:167-175 (1995)), GAGE family antigens (i.e., GAGE-1,2; Van den Eynde et al., J. Exp. Med., 182:689-698 (1995); U.S. Pat. No. 6,013,765), RAGE family antigens (i.e., RAGE-1; Gaugler et at., Immunogenetics, 44:323-330 (1996); U.S. Pat. No. 5,939,526), N-acetylglucosaminyltransferase-V (Guilloux et at., J. Exp. Med., 183:1173-1183 (1996)), p15 (Robbins et al., J. Immunol. 154:5944-5950 (1995)), β-catenin (Robbins et al., J. Exp. Med., 183:1185-1192 (1996)), MUM-1 (Coulie et al., Proc. Natl. Acad. Sci. USA, 92:7976-7980 (1995)), cyclin dependent kinase-4 (CDK4) (Wolfel et al., Science, 269:1281-1284 (1995)), p21-ras (Fossum et at., Int. J. Cancer, 56:40-45 (1994)), BCR-abl (Bocchia et al., Blood, 85:2680-2684 (1995)), p53 (Theobald et al., Proc. Natl. Acad. Sci. USA, 92:11993-11997 (1995)), p185 HER2/neu (erb-B1; Fisk et al., J. Exp. Med., 181:2109-2117 (1995)), epidermal growth factor receptor (EGFR) (Harris et al., Breast Cancer Res. Treat, 29:1-2 (1994)), carcinoembryonic antigens (CEA) (Kwong et al., J. Natl. Cancer Inst., 85:982-990 (1995) U.S. Pat. Nos. 5,756,103; 5,274,087; 5,571,710; 6,071,716; 5,698,530; 6,045,802; EP 263933; EP 346710; and, EP 784483); carcinoma-associated mutated mucins (i.e., MUC-1 gene products; Jerome et al., J. Immunol., 151:1654-1662 (1993)); EBNA gene products of EBV (i.e., EBNA-1; Rickinson et al., Cancer Surveys, 13:53-80 (1992)); E7, E6 proteins of human papillomavirus (Ressing et al., J. Immunol, 154:5934-5943 (1995)); prostate specific antigen (PSA; Xue et al., The Prostate, 30:73-78 (1997)); prostate specific membrane antigen (PSMA; Israeli, et al., Cancer Res., 54:1807-1811 (1994)); idiotypic epitopes or antigens, for example, immunoglobulin idiotypes or T cell receptor idiotypes (Chen et al., J. Immunol., 153:4775-4787 (1994)); KSA (U.S. Pat. No. 5,348,887), kinesin 2 (Dietz, et al. Biochem Biophys Res Commun Sep. 7, 2000;275(3):731-8), HIP-55, TGFβ-1 anti-apoptotic factor (Toomey, et al. Br J Biomed Sci 2001;58(3):177-83), tumor protein D52 (Bryne J. A., et al., Genomics, 35:523-532 (1996)), HIFT, NY-BR-1 (WO 01/47959), NY-BR-62, NY-BR-75, NY-BR-85, NY-BR-87 and NY-BR-96 (Scanlan, M. Serologic and Bioinformatic Approaches to the Identification of Human Tumor Antigens, in Cancer Vaccines 2000, Cancer Research Institute, New York, N.Y.), including wild-type, modified, mutated TAs as well as immunogenic fragments and derivatives thereof Any of these TAs may be utilized alone or in combination with one or more targeted immunogens in a co-immunization protocol.

Many suitable TA-derived peptide sequences are suitable for use in practicing the present invention. Preferred TA-derived peptide sequences, any of which may be joined to a targeting sequence such as such as TAT, AntP, hPER1-1 or hPER1-2, are shown below:

| | | |
|---|---|---|
| gp100-280-288(9V) | YLEPGPVTV | (SEQ ID NO:5) |
| gp100-154-162 | KTWGQYWQV | (SEQ ID NO:6) |
| MART-1 32 | ILTVILGVL | (SEQ. ID. NO.7) |
| MART-1 31 | GILTVILGV | (SEQ. ID. NO.8) |
| MART-1 99 | NAPPAYEKL | (SEQ. ID. NO.9) |
| MART-1 1 | MPREDAHFI | (SEQ. ID. NO.10) |
| MART-1 56 | ALMDKSLHV | (SEQ ID. NO.11) |
| MART-1 39 | VLLLIGCWY | (SEQ. ID. NO.12) |
| MART-1 35 | VILGVLLLI | (SEQ. ID. NO.13) |
| MART-1 61 | SLHVGTQCA | (SEQ. ID. NO.14) |
| MART-1 57 | LMDKSLHVG | (SEQ. ID. NO.15) |
| MAGE-A3 115 | ELVHFLLLK | (SEQ ID NO:16) |
| MAGE-A3 285 | KVLHHMVKI | (SEQ ID NO:17) |
| MAGE-A3 276 | RALVETSYV | (SEQ ID NO:18) |
| MAGE-A3 105 | FQAALSRKV | (SEQ ID NO:19) |
| MAGE-A3 296 | GPHISYPPL | (SEQ ID NO:20) |
| MAGE-A3 243 | KKLLTQHFV | (SEQ ID NO.21) |
| MAGE-A3 24 | GLVGAQAPA | (SEQ ID NO.22) |
| MAGE-A3 301 | YPPLHEWVL | (SEQ ID NO.23) |
| MAGE-A3 71 | LPTTMNYPL | (SEQ ID NO.24) |
| Tyr 171 | NIYDLFVWM | (SEQ ID NO:25) |
| Tyr 444 | DLGYDYSYL | (SEQ ID NO:26) |
| Tyr 57 | NILLSNAPL | (SEQ ID NO:27) |
| TRP-1 245 | SLPYWNFAT | (SEQ ID NO:28) |
| TRP-1 298 | TLGTLCNST | (SEQ ID NO:29) |
| TRP-1 481 | IAVVGALLL | (SEQ ID NO:30) |
| TRP-1 181 | NISIYNYFV | (SEQ ID NO:31) |
| TRP-1 439 | NMVPFWPPV | (SEQ ID NO:32) |

In certain embodiments, the targeting sequences may be joined to immunogenic peptide sequences with a linker sequence inserted between the targeting sequence and the immunogenic sequence. Suitable linkers include, for example, amino acid sequences naturally occur with N-terminal to the N-terminus of the peptide sequence in the full-length parental polypeptide from which the peptide was derived. For example, the gp100 peptide sequence TWGQYWQV naturally occurs with the sequence FVYVW at its N-terminus within the full-length gp100 polypeptide. Accordingly, FVYVW may serve to link the gp100 peptide to a targeting sequence. Other suitable linkers may be devised using standard methods for designing peptides that interact with MHC molecules, as is known in the art.

Derivatives of the peptide sequences of the present invention may also be in certain embodiments. One type of derivative is a sequence in which one amino acid sequence is substituted by another. Substitutions may be conservative, or non-conservative, or any combination thereof. Conservative amino acid modifications to the sequence of a polypeptide (and the corresponding modifications to the encoding nucleotides) may produce polypeptides having functional and chemical characteristics similar to those of a parental polypeptide. For example, a “conservative amino acid substitution” may involve a substitution of a native amino acid residue with a non-native residue such that there is little or no effect on the size, polarity, charge, hydrophobicity, or hydrophilicity of the amino acid residue at that position and, in particlar, does not result in decreased immunogenicity. Suitable conservative amino acid substitutions are shown in Table I.

TABLE I

| Original | | Preferred |
|---|---|---|
| Residues | Exemplary Substitutions | Substitutions |
| Ala | Val, Leu, Ile | Val |
| Arg | Lys, Gln, Asn | Lys |
| Asn | Gln | Gln |
| Asp | Glu | Glu |
| Cys | Ser, Ala | Ser |
| Gln | Asn | Asn |
| Glu | Asp | Asp |
| Gly | Pro, Ala | Ala |
| His | Asn, Gln, Lys, Arg | Arg |
| Ile | Leu, Val, Met, Ala, Phe, Norleucine | Leu |
| Leu | Norleucine, Ile, Val, Met, Ala, Phe | Ile |
| Lys | Arg, 1,4 Diamino-butyric Acid, Gln, Asn | Arg |
| Met | Leu, Phe, Ile | Leu |
| Phe | Leu, Val, Ile, Ala, Tyr | Leu |
| Pro | Ala | Gly |
| Ser | Thr, Ala, Cys | Thr |
| Thr | Ser | Ser |
| Trp | Tyr, Phe | Tyr |
| Tyr | Trp, Phe, Thr, Ser | Phe |
| Val | Ile, Met, Leu, Phe, Ala, Norleucine | Leu |

A skilled artisan will be able to determine suitable variants of an immunogenic target using well-known techniques. For identifying suitable areas of the molecule that may be changed without destroying biological activity (i.e., MHC binding, immunogenicity), one skilled in the art may target areas not believed to be important for that activity. For example, when immunogenic targets with similar activities from the same species or from other species are known, one skilled in the art may compare the amino acid sequence of a polypeptide to such similar polypeptides. By performing such analyses, one can identify residues and portions of the molecules that are conserved. It will be appreciated that changes in areas of the molecule that are not conserved relative to such similar immunogenic targets would be less likely to adversely affect the biological activity and/or structure of a polypeptide. One skilled in the art would also know that, even in relatively conserved regions, one may substitute chemically similar amino acids for the naturally occurring residues while retaining activity. Therefore, even areas that may be important for biological activity or for structure may be subject to conservative amino acid substitutions without destroying the biological activity or without adversely affecting the structure of the immunogenic target.

In certain embodiments, a nucleic acid molecule encoding the peptide sequences may be inserted into expression vectors, as discussed below in greater detail. In such embodiments, the peptide sequences are encoded by nucleotides corresponding to the amino acid sequence. The particular combinations of nucleotides that encode the various amino acids are well known in the art, as described in various references used by those skilled in the art (i.e., Lewin, B. Genes V, Oxford University Press, 1994), as shown in Table II below:

TABLE II

| | | | | | | | |
|---|---|---|---|---|---|---|---|
| Phe | TTT | Ser | TCT | Tyr | TAT | Cys | TGT |
| | TTC | | TCC | | TAC | | TGC |
| Leu | TTA | | TCA | TERM | TAA | TERM | TGA |
| | TTG | | TCG | | TAG | Trp | TGG |
| | CTT | Pro | CCT | His | CAT | Arg | CGT |
| | CTC | | CCC | | CAC | | CGC |
| | CTA | | CCA | Gin | CAA | | CGA |
| | CTG | | CCG | | CAG | | CGG |
| Ile | ATT | Thr | ACT | Asn | AAT | Ser | AGT |
| | ATC | | ACC | | AAC | | AGC |
| | ATA | | ACA | Lys | AAA | Arg | AGA |
| Met | ATG | | ACG | | AAG | | AGG |
| Val | GTT | Ala | GCT | Asp | GAT | Gly | GGT |
| | GTC | | GCC | | GAC | | GGC |
| | GTA | | GCA | Glu | GAA | | GGA |
| | GTG | | GCG | | GAG | | GGG |

Exemplary DNA sequences encoding the various peptides of the present invention are shown below:

| | | |
|---|---|---|
| TAT: | GGCTACGGCAGGAAGAAGAGGAGGCAGAGGAGGAGG | (SEQ ID NO.:33) |
| AntP: | AGGCAGATCAAGATCTGGTTCCAGAACAGGAGGATGAAGTGGAAGAAG | (SEQ ID NO.:34) |
| PER1-1: | AGCAGGAGGCACCACTGCAGGAGCAAGGCCAAGAGGAGCAGGCACCAC | (SEQ ID NO.:35) |
| PER1-2: | GGCAGGAGGCACCACAGGAGGAGCAAGGCCAAGAGGAGCAGG | (SEQ ID NO.:36) |
| gp100-280-288(9V): | TACCTGGAGCCCGGCCCCGTGACCGTG | (SEQ ID NO.:37) |
| gp100-154-162: | AAGACCTGGGGCCAGTACTGGCAGGTG | (SEQ ID NO.:38) |
| MART-1 32: | ATCCTGACAGTGATCCTGGGAGTCTTA | (SEQ ID NO:39) |
| MART-1 31: | GGCATCCTGACAGTGATCCTGGGAGTC | (SEQ ID NO:40) |
| MART-1 99: | AATGCTCCACCTGCTTATGAGAAACTC | (SEQ ID NO:42) |
| MART-1 1: | ATGCCAAGAGAAGATGCTCACTTCATC | (SEQ ID NO:43) |
| MART-1 56: | GCCTTGATGGATAAAAGTCTTCATGTT | (SEQ ID NO:44) |
| MART-1 39: | GTCTTACTGCTCATCGGCTGTTGGTAT | (SEQ ID NO:45) |
| MART-1 35: | GTGATCCTGGGAGTCTTACTGCTCATC | (SEQ ID NO:46) |
| MART-1 61: | AGTCTTCATGTTGGCACTCAATGTGCC | (SEQ ID NO:47) |
| MART-1 57: | TTGATGGATAAAAGTCTTCATGTTGGC | (SEQ ID NO:48) |
| MAGE-A3 115: | GAGTTGGTTCATTTTCTGCTCCTCAAG | (SEQ ID NO.49) |
| MAGE-A3 285: | AAAGTCCTGCACCATATGGTAAAGATC | (SEQ.ID.NO.50) |
| MAGE-A3 276: | AGGGCCCTCGTTGAAACCAGCTATGTG | (SEQ ID.NO.51) |
| MAGE-A3 105: | TTCCAAGCAGCACTCAGTAGGAAGGTG | (SEQ ID.NO.52) |
| MAGE-A3 296: | GGACCTCACATTTCCTACCCACCCCTG | (SEQ.ID.NO.53) |
| MAGE-A3 243: | AAGAAGCTGCTCACCCAACATTTCGTG | (SEQ ID.NO.54) |
| MAGE-A3 24: | GGCCTGGTGGGTGCGCAGGCTCCTGCT | (SEQ ID NO:55) |
| MAGE-A3 301: | TACCCACCCCTGCATGAGTGGGTTTTG | (SEQ ID.NO.56) |
| MAGE-A3 71: | CTCCCCACTACCATGAACTACCCTCTC | (SEQ.ID.NO.57) |
| TYR 171: | AATATTTATGACCTCTTTGTCTGGATG | (SEQ ID NO:58) |
| TYR 444: | GATCTGGGCTATGACTATAGCTATCTA | (SEQ ID NO:59) |
| TYR 57: | AATATCCTTCTGTCCAATGCACCACTT | (SEQ ID NO:60) |
| TRP-1 245: | TCCCTTCCTTACTGGAATTTTGCAACG | (SEQ ID NO:61) |
| TRP-1 298: | ACCCTGGGAACACTTTGTAACAGCACC | (SEQ ID NO:62) |
| TRP-1 481: | ATAGCAGTAGTTGGCGCTTTGTTACTG | (SEQ ID NO:63) |
| TRP-1 181: | AACATTTCCATTTATAACTACTTTGTT | (SEQ ID NO:64) |
| TRP-1 439: | AACATGGTGCCATTCTGGCCCCCAGTC | (SEQ ID NO:65) |

Shown below are amino acid and DNA sequences of exemplary immunogenic targets including a first amino acid representing a targeting sequence and a second amino acid sequence representing an immunogen (T cell epitope):

| | |
|---|---|
| hPER1-1-gp100 (280-288) | |
| S R R H H C R S K A K R S R H | |
| AGC AGG AGG CAC CAC TGC AGG AGC AAG GCC AAG AGG AGC AGG CAC | (SEQ ID NO:66) |
| H Y L E P G P V T V | |
| CAC TAC CTG GAG CCC GGC CCC GTG ACC GTG | |
| hPER1-2-gp100 (154-162) | |
| G R R H H R R S K A K R S R A | |
| GGC AGG AGG CAC CAC AGG AGG AGC AAG GCC AAG AGG AGC AGG GCC | (SEQ ID NO:67) |
| S N E N M E T M K T W G Q Y W | |
| AGC AAC GAG AAC ATG GAG ACC ATG AAG ACC TGG GGC CAG TAC TGG | |
| Q V | |
| CAG GTG | (SEQ ID NO:67) |
| hPER1-2-F-gp100 (154-162) | |
| G R R H H R R S K A K R S R A | |
| GGC AGG AGG CAC CAC AGG AGG AGC AAG GCC AAG AGG AGC AGG GCC | (SEQ ID NO:68) |
| S N E N M E T M F V Y V W K T | |
| AGC AAC GAG AAC ATG GAG ACC ATG TTC GTG TAC GTG TGG AAG ACC | (SEQ ID NO:68) |
| W G Q Y W Q V | |
| TGG GGC CAG TAC TGG CAG GTG | |

A targeted immunogen may be administered in combination with adjuvants and/or cytokines to boost the immune response. Exemplary adjuvants are shown in Table III below:

TABLE III
Types of Immunologic Adjuvants

| Type of | | |
|---|---|---|
| Adjuvant | General Examples | Specific Examples/References |
| Gel-type | Aluminum hydroxide/phosphate (“alum | (Aggerbeck and Heron, 1995) |
| | adjuvants”) | |
| | Calcium phosphate | (Relyveld, 1986) |
| Microbial | Muramyl dipeptide (MDP) | (Chedid et al., 1986) |
| | Bacterial exotoxins | Cholera toxin (CT), E. coli labile toxin |
| | | (LT) (Freytag and Clements, 1999) |
| | Endotoxin-based adjuvants | Monophosphoryl lipid A (MPL) |
| | | (Ulrich and Myers, 1995) |
| | Other bacterial | CpG oligonucleotides (Corral and |
| | | Petray, 2000), BCG sequences (Krieg, et |
| | | al. Nature, 374:576), tetanus toxoid |
| | | (Rice, et al. J. Immunol., 2001, 167: |
| | | 1558-1565) |
| Particulate | Biodegradable | (Gupta et al., 1998) |
| | Polymer microspheres | |
| | Immunostimulatory complexes | (Morein and Bengtsson, 1999) |
| | (ISCOMs) | |
| | Liposomes | (Wassef et al., 1994) |
| Oil-emulsion | Freund's incomplete adjuvant | (Jensen et al., 1998) |
| and | Microfluidized emulsions | MF59 (Ott et al., 1995) |
| surfactant- | | SAF (Allison and Byars, 1992) |
| based | | (Allison, 1999) |
| adjuvants | Saponins | QS-21 (Kensil, 1996) |
| Synthetic | Muramyl peptide derivatives | Murabutide (Lederer, 1986) |
| | | Threony-MDP (Allison, 1997) |
| | Nonionic block copolymers | L121 (Allison, 1999) |
| | Polyphosphazene (PCPP) | (Payne et al., 1995) |
| | Synthetic polynucleotides | Poly A:U, Poly I:C (Johnson, 1994) |
| | Thalidomide derivatives | CC-4047/ACTIMID (J. Immunol., |
| | | 168(10):4914-9) |

One or more cytokines may also be suitable co-stimulatory components in practicing the present invention, either as polypeptides or as encoded by nucleic acids contained within the compositions of the present invention (Parmiani, et al. Immunol Lett Sep. 15, 2000; 74(1): 41-4; Berzofsky, et al. Nature Immunol. 1: 209-219). Suitable cytokines include, for example, interleukin-2 (IL-2) (Rosenberg, et al. Nature Med. 4: 321-327 (1998)), IL-4, IL-7, IL-12 (reviewed by Pardoll, 1992; Harries, et al. J. Gene Med. 2000 July-August;2(4):243-9; Rao, et al. J. Immunol. 156: 3357-3365 (1996)), IL-15 (Xin, et al. Vaccine, 17:858-866, 1999), IL-16 (Cruikshank, et al. J. Leuk Biol. 67(6): 757-66, 2000), IL-18 (J. Cancer Res. Clin. Oncol. 2001. 127(12): 718-726), GM-CSF (CSF (Disis, et al. Blood, 88: 202-210 (1996)), tumor necrosis factor-alpha (TNF-α), or interferon-gamma (INF-γ). Other cytokines may also be suitable for practicing the present invention, as is known in the art.

Chemokines may also be used to assist in inducing or enhancing the immune response. For example, fusion proteins comprising CXCL10 (IP-10) and CCL7 (MCP-3) fused to a tumor self-antigen have been shown to induce anti-tumor immunity (Biragyn, et al. Nature Biotech. 1999, 17: 253-258). The chemokines CCL3 (MIP-1α) and CCL5 (RANTES) (Boyer, et al. Vaccine, 1999, 17 (Supp. 2): S53-S64) may also be of use in practicing the present invention. Other suitable chemokines are known in the art.

In certain embodiments, the targeted immunogen may be utilized as a nucleic acid molecule, either alone or as part of a delivery vehicle such as a viral vector. In such cases, it may be advantageous to combine the targeted immunogen with one or more co-stimulatory component(s) such as cell surface proteins, cytokines or chemokines in a composition of the present invention. The co-stimulatory component may be included in the composition as a polypeptide or as a nucleic acid encoding the polypeptide, for example. Suitable co-stimulatory molecules include, for instance, polypeptides that bind members of the CD28 family (i.e., CD28, ICOS; Hutloff, et al. Nature 1999, 397: 263-265; Peach, et al. J Exp Med 1994, 180: 2049-2058) such as the CD28 binding polypeptides B7.1 (CD80; Schwartz, 1992; Chen et al, 1992; Ellis, et al. J. Immunol., 156(8): 2700-9) and B7.2 (CD86; Ellis, et al. J. Immunol., 156(8): 2700-9); polypeptides which bind members of the integrin family (i.e., LFA-1 (CD11a/CD18); Sedwick, et al. J Immunol 1999, 162: 1367-1375; Wülfing, et al. Science 1998, 282: 2266-2269; Lub, et al. Immunol Today 1995, 16: 479-483) including members of the ICAM family (i.e., ICAM-1, -2 or -3); polypeptides which bind CD2 family members (i.e., CD2, signalling lymphocyte activation molecule (CDw150 or “SLAM”; Aversa, et al. J Immunol 1997, 158: 4036-4044) such as CD58 (LFA-3; CD2 ligand; Davis, et al. Immunol Today 1996, 17: 177-187) or SLAM ligands (Sayos, et al. Nature 1998, 395: 462-469); polypeptides which bind heat stable antigen (HSA or CD24; Zhou, et al. Eur J Immunol 1997, 27: 2524-2528); polypeptides which bind to members of the TNF receptor (TNFR) family (i.e., 4-1BB (CD137; Vinay, et al. Semin Immunol 1998, 10: 481-489)), OX40 (CD134; Weinberg, et al. Semin Immunol 1998, 10: 471-480; Higgins, et al. J Immunol 1999, 162: 486-493), and CD27 (Lens, et al. Semin Immunol 1998, 10: 491-499)) such as 4-1BBL (4-1BB ligand; Vinay, et al. Semin Immunol 1998, 10: 481-48; DeBenedette, et al. J Immunol 1997, 158: 551-559), TNFR associated factor-1 (TRAF-1; 4-1BB ligand; Saoulli, et al. J Exp Med 1998, 187: 1849-1862, Arch, et al. Mol Cell Biol 1998, 18: 558-565), TRAF-2 (4-1BB and OX40 ligand; Saoulli, et al. J Exp Med 1998, 187: 1849-1862; Oshima, et al. Int Immunol 1998, 10: 517-526, Kawamata, et al. J Biol Chem 1998, 273: 5808-5814), TRAF-3 (4-1BB and OX40 ligand; Arch, et al. Mol Cell Biol 1998, 18: 558-565; Jang, et al. Biochem Biophys Res Commun 1998, 242: 613-620; Kawamata S, et al. J Biol Chem 1998, 273: 5808-5814), OX40L (OX40 ligand; Gramaglia, et al. J Immunol 1998, 161: 6510-6517), TRAF-5 (OX40 ligand; Arch, et al. Mol Cell Biol 1998, 18: 558-565; Kawamata, et al. J Biol Chem 1998, 273: 5808-5814), and CD70 (CD27 ligand; Couderc, et al. Cancer Gene Ther., 5(3): 163-75). CD154 (CD40 ligand or “CD40L”; Gurunathan, et al. J. Immunol., 1998, 161: 4563-4571; Sine, et al. Hum. Gene Ther., 2001, 12: 1091-1102) may also be suitable. Stimulatory motifs other than co-stimulatory molecules per se may be incorporated into nuclec acids encoding TAs, such as CpG motifs (Gurunathan, et al. Ann. Rev. Immunol., 2000, 18: 927-974). Other stimulatory motifs or co-stimulatory molecules may also be useful in treating and/or preventing cancer, using the reagents and methodologies herein described.

Any of these co-stimulatory components may be used alone or in combination with other agents. For instance, it has been shown that a combination of CD80, ICAM-1 and LFA-3 (“TRICOM”) may potentiate anti-cancer immune responses (Hodge, et al. Cancer Res. 59: 5800-5807 (1999). Other effective combinations include, for example, IL-12+GM-CSF (Ahlers, et al. J. Immunol., 158: 3947-3958 (1997); Iwasaki, et al. J. Immunol. 158: 4591-4601 (1997)), IL-12+GM-CSF+TNF-α (Ahlers, et al. Int. Immunol. 13: 897-908 (2001)), CD80+IL-12 (Fruend, et al. Int. J. Cancer, 85: 508-517 (2000); Rao, et al. supra), and CD86+GM-CSF +IL-12 (Iwasaki, supra). One of skill in the art would be aware of additional combinations useful in carrying out the present invention.

It is also known in the art that suppressive or negative regulatory immune mechanisms may be blocked, resulting in enhanced immune responses. For instance, treatment with anti-CTLA-4 (Shrikant, et al. Immunity, 1996, 14: 145-155; Sutmuller, et al. J Exp. Med., 2001, 194: 823-832), anti-CD25 (Sutmuller, supra), anti-CD4 (Matsui, et al. J. Immunol., 1999, 163: 184-193), the fusion protein IL13Ra2-Fc (Terabe, et al. Nature Immunol., 2000, 1: 515-520), and combinations thereof (i.e., anti-CTLA-4 and anti-CD25, Sutmuller, supra) have been shown to upregulate anti-tumor immune responses. In addition, the skilled artisan would be aware of additional reagents or methods that may be used to modulate such mechanisms. These reagents and methods, as well as others known by those of skill in the art, may be utilized in practicing the present invention.

Expression vectors may also be suitable for use in practicing the present invention. Expression vectors are typically comprised of a flanking sequence operably linked to a heterologous nucleic acid sequence encoding a polypeptide (the “coding sequence”). In preferred embodiments, the polypeptide consists of a first amino acid sequence representing a targeting sequence and a second amino acid sequence representing an immunogen (i.e., a T cell epitope). A flanking sequence is preferably capable of effecting the replication, transcription and/or translation of the coding sequence and is operably linked to a coding sequence. To be “operably linked” indicates that the nucleic acid sequences are configured so as to perform their usual function. For example, a promoter is operably linked to a coding sequence when the promoter is capable of directing transcription of that coding sequence. A flanking sequence need not be contiguous with the coding sequence, so long as it functions correctly. Thus, for example, intervening untranslated yet transcribed sequences can be present between a promoter sequence and the coding sequence and the promoter sequence can still be considered operably linked to the coding sequence. Flanking sequences may be homologous (i.e., from the same species and/or strain as the host cell), heterologous (i.e., from a species other than the host cell species or strain), hybrid (i.e., a combination of flanking sequences from more than one source), or synthetic. A flanking sequence may also be a sequence that normally functions to regulate expression of the nucleotide sequence encoding the polypeptide in the genome of the host may also be utilized.

In certain embodiments, it is preferred that the flanking sequence is a transcriptional regulatory region that drives high-level gene expression in the target cell. The transcriptional regulatory region may comprise, for example, a promoter, enhancer, silencer, repressor element, or combinations thereof. The transcriptional regulatory region may be either constitutive or tissue- or cell-type specific (i.e., the region is drives higher levels of transcription in a one type of tissue or cell as compared to another). As such, the source of a transcriptional regulatory region may be any prokaryotic or eukaryotic organism, any vertebrate or invertebrate organism, or any plant, provided that the flanking sequence is functional in, and can be activated by, the host cell machinery. A wide variety of transcriptional regulatory regions may be utilized in practicing the present invention.

Suitable transcriptional regulatory regions include, among others, the CMV promoter (i.e., the CMV-immediate early promoter); promoters from eukaryotic genes (i.e., the estrogen-inducible chicken ovalbumin gene, the interferon genes, the gluco-corticoid-inducible tyrosine aminotransferase gene, and the thymidine kinase gene); and the major early and late adenovirus gene promoters; the SV40 early promoter region (Bernoist and Chambon, 1981, Nature 290:304-10); the promoter contained in the 3′ long terminal repeat (LTR) of Rous sarcoma virus (RSV) (Yamamoto, et al., 1980, Cell 22:787-97); the herpes simplex virus thymidine kinase (HSV-TK) promoter (Wagner et al., 1981, Proc. Natl. Acad. Sci. U.S.A. 78:1444-45); the regulatory sequences of the metallothionine gene (Brinster et al., 1982, Nature 296:39-42); prokaryotic expression vectors such as the beta-lactamase promoter (Villa-Kamaroff et al., 1978, Proc. Natl. Acad. Sci. U.S.A., 75:3727-31); or the tac promoter (DeBoer et al., 1983, Proc. Natl. Acad. Sci. U.S.A., 80:21-25). Tissue- and/or cell-type specific transcriptional control regions include, for example, the elastase I gene control region which is active in pancreatic acinar cells (Swift et al., 1984, Cell 38:639-46; Omitz et al., 1986, Cold Spring Harbor Symp. Quant. Biol. 50:399-409 (1986); MacDonald, 1987, Hepatology 7:425-515); the insulin gene control region which is active in pancreatic beta cells (Hanahan, 1985, Nature 315:115-22); the immunoglobulin gene control region which is active in lymphoid cells (Grosschedl et al., 1984, Cell 38:647-58; Adames et al., 1985, Nature 318:533-38; Alexander et al., 1987, Mol. Cell. Biol., 7:1436-44); the mouse mammary tumor virus control region in testicular, breast, lymphoid and mast cells (Leder et al., 1986, Cell 45:485-95); the albumin gene control region in liver (Pinkert et al., 1987, Genes and Devel. 1:268-76); the alpha-feto-protein gene control region in liver (Krumlauf et al., 1985, Mol. Cell. Biol., 5:1639-48; Hammer et al., 1987, Science 235:53-58); the alpha 1-antitrypsin gene control region in liver (Kelsey et al, 1987, Genes and Devel. 1:161-71); the beta-globin gene control region in myeloid cells (Mogram et al., 1985, Nature 315:338-40; Kollias et al., 1986, Cell 46:89-94); the myelin basic protein gene control region in oligodendrocyte cells in the brain (Readhead et al., 1987, Cell 48:703-12); the myosin light chain-2 gene control region in skeletal muscle (Sani, 1985, Nature 314:283-86); and the gonadotropic releasing hormone gene control region in the hypothalamus (Mason et al., 1986, Science 234:1372-78), and the tyrosinase promoter in melanoma cells (Hart, I. Semin Oncol 1996 February;23(1):154-8; Siders, et al. Cancer Gene Ther 1998 September-October;5(5):281-91). Other suitable promoters are known in the art.

The nucleic acid molecule encoding the targeted immunogen may be administered as part of a viral and non-viral vector. In one embodiment, a DNA vector is utilized to deliver nucleic acids encoding the targeted immunogen and/or associated molecules (i.e., co-stimulatory molecules, cytokines or chemokines) to the patient. In doing so, various strategies may be utilized to improve the efficiency of such mechanisms including, for example, the use of self-replicating viral replicons (Caley, et al. 1999. Vaccine, 17: 3124-2135; Dubensky, et al. 2000. Mol. Med. 6: 723-732; Leitner, et al. 2000. Cancer Res. 60: 51-55), codon optimization (Liu, et al. 2000. Mol. Ther., 1: 497-500; Dubensky, supra; Huang, et al. 2001. J. Virol. 75: 4947-4951), in vivo electroporation (Widera, et al. 2000. J. Immunol. 164: 4635-3640), incorporation of nucleic acids encoding co-stimulatory molecules, cytokines and/or chemokines (Xiang, et al. 1995. Immunity, 2: 129-135; Kim, et al. 1998. Eur. J. Immunol., 28: 1089-1103; Iwasaki, et al. 1997. J. Immunol. 158: 4591-4601; Sheerlinck, et al. 2001. Vaccine, 19: 2647-2656), incorporation of stimulatory motifs such as CpG (Gurunathan, supra; Leitner, supra), sequences for targeting of the endocytic or ubiquitin-processing pathways (Thomson, et al. 1998. J. Virol. 72: 2246-2252; Velders, et al. 2001. J. Immunol. 166: 5366-5373), prime-boost regimens (Gurunathan, supra; Sullivan, et al. 2000. Nature, 408: 605-609; Hanke, et al. 1998. Vaccine, 16: 439-445; Amara, et al. 2001. Science, 292: 69-74), proteasome-sensitive cleavage sites, and the use of mucosal delivery vectors such as Salmonella (Darji, et al. 1997. Cell, 91: 765-775; Woo, et al. 2001. Vaccine, 19: 2945-2954). Other methods are known in the art, some of which are described below.

Various viral vectors that have been successfully utilized for introducing a nucleic acid to a host include retrovirus, adenovirus, adeno-associated virus (AAV), herpes virus, and poxvirus, among others. It is understood in the art that many such viral vectors are available in the art. The vectors of the present invention may be constructed using standard recombinant techniques widely available to one skilled in the art. Such techniques may be found in common molecular biology references such as Molecular Cloning: A Laboratory Manual (Sambrook, et al., 1989, Cold Spring Harbor Laboratory Press), Gene Expression Technology (Methods in Enzymology, Vol. 185, edited by D. Goeddel, 1991. Academic Press, San Diego, Calif.), and PCR Protocols: A Guide to Methods and Applications (Innis, et al. 1990. Academic Press, San Diego, Calif.).

Preferred retroviral vectors are derivatives of lentivirus as well as derivatives of murine or avian retroviruses. Examples of suitable retroviral vectors include, for example, Moloney murine leukemia virus (MoMuLV), Harvey murine sarcoma virus (HaMuSV), murine mammary tumor virus (MuMTV), SIV, BIV, HIV and Rous Sarcoma Virus (RSV). A number of retroviral vectors can incorporate multiple exogenous nucleic acid sequences. As recombinant retroviruses are defective, they require assistance in order to produce infectious vector particles. This assistance can be provided by, for example, helper cell lines encoding retrovirus structural genes. Suitable helper cell lines include Ψ2, PA317 and PA12, among others. The vector virions produced using such cell lines may then be used to infect a tissue cell line, such as NIH 3T3 cells, to produce large quantities of chimeric retroviral virions. Retroviral vectors may be administered by traditional methods (i.e., injection) or by implantation of a “producer cell line” in proximity to the target cell population (Culver, K., et al., 1994, Hum. Gene Ther., 5 (3): 343-79; Culver, K., et al., Cold Spring Harb. Symp. Quant. Biol., 59: 685-90); Oldfield, E., 1993, Hum. Gene Ther., 4 (1): 39-69). The producer cell line is engineered to produce a viral vector and releases viral particles in the vicinity of the target cell. A portion of the released viral particles contact the target cells and infect those cells, thus delivering a nucleic acid of the present invention to the target cell. Following infection of the target cell, expression of the nucleic acid of the vector occurs.

Adenoviral vectors have proven especially useful for gene transfer into eukaryotic cells (Rosenfeld, M., et al., 1991, Science, 252 (5004): 431-4; Crystal, R., et al., 1994, Nat. Genet., 8 (1): 42-51), the study eukaryotic gene expression (Levrero, M., et al., 1991, Gene, 101 (2): 195-202), vaccine development (Graham, F. and Prevec, L., 1992, Biotechnology, 20: 363-90), and in animal models (Stratford-Perricaudet, L., et al., 1992, Bone Marrow Transplant., 9 (Suppl. I): 151-2; Rich, D., et al., 1993, Hum. Gene Ther., 4 (4): 461-76). Experimental routes for administrating recombinant Ad to different tissues in vivo have included intratracheal instillation (Rosenfeld, M., et al., 1992, Cell, 68 (1): 143-55) injection into muscle (Quantin, B., et al., 1992, Proc. Natl. Acad. Sci. U.S.A., 89 (7): 2581-4), peripheral intravenous injection (Herz, J., and Gerard, R., 1993, Proc. Natl. Acad. Sci. U.S.A., 90 (7): 2812-6) and stereotactic inoculation to brain (Le Gal La Salle, G., et al., 1993, Science, 259 (5097): 988-90), among others.

Adeno-associated virus (AAV) demonstrates high-level infectivity, broad host range and specificity in integrating into the host cell genome (Hernonat, P., et al., 1984, Proc. Natl. Acad. Sci. U.S.A., 81 (20): 6466-70). And Herpes Simplex Virus type-I (HSV-1) is yet another attractive vector system, especially for use in the nervous system because of its neurotropic property (Geller, A., et al., 1991, Trends Neurosci., 14 (10): 428-32; Glorioso, et al., 1995, Mol. Biotechnol., 4 (1): 87-99; Glorioso, et al., 1995, Annu. Rev. Microbiol., 49: 675-710).

Poxvirus is another useful expression vector (Smith, et al. 1983, Gene, 25 (1): 21-8; Moss, et al, 1992, Biotechnology, 20: 345-62; Moss, et al, 1992, Curr. Top. Microbiol. Immunol., 158: 25-38; Moss, et al. 1991. Science, 252: 1662-1667). Poxviruses shown to be useful include vaccinia, NYVAC, avipox, fowlpox, canarypox, ALVAC, and ALVAC(2), among others.

NYVAC (vP866) was derived from the Copenhagen vaccine strain of vaccinia virus by deleting six nonessential regions of the genome encoding known or potential virulence factors (see, for example, U.S. Pat. Nos. 5,364,773 and 5,494,807). The deletion loci were also engineered as recipient loci for the insertion of foreign genes. The deleted regions are: thymidine kinase gene (TK; J2R) vP410; hemorrhagic region (u; B13R+Bl4R) vP553; A type inclusion body region (AT1; A26L) vP618; hemagglutinin gene (HA; A56R) vP723; host range gene region (C7L-K1L) vP804; and, large subunit, ribonucleotide reductase (14L) vP866. NYVAC is a genetically engineered vaccinia virus strain that was generated by the specific deletion of eighteen open reading frames encoding gene products associated with virulence and host range. NYVAC has been show to be useful for expressing TAs (see, for example, U.S. Pat. No. 6,265,189). NYVAC (vP866), vP994, vCP205, vCP1433, placZH6H4Lreverse, pMPC6H6K3E3 and pC3H6FHVB were also deposited with the ATCC under the terms of the Budapest Treaty, accession numbers VR-2559, VR-2558, VR-2557, VR-2556, ATCC-97913, ATCC-97912, and ATCC-97914, respectively.

ALVAC-based recombinant viruses (i.e., ALVAC-1 and ALVAC-2) are also suitable for use in practicing the present invention (see, for example, U.S. Pat. No. 5,756,103). ALVAC(2) is identical to ALVAC(1) except that ALVAC(2) genome comprises the vaccinia E3L and K3L genes under the control of vaccinia promoters (U.S. Pat. No. 6,130,066; Beattie et al., 1995a, 1995b, 1991; Chang et al., 1992; Davies et al., 1993). Both ALVAC(1) and ALVAC(2) have been demonstrated to be useful in expressing foreign DNA sequences, such as TAs (Tartaglia et al., 1993 a,b; U.S. Pat. No. 5,833,975). ALVAC was deposited under the terms of the Budapest Treaty with the American Type Culture Collection (ATCC), 10801 University Boulevard, Manassas, Va. 20110-2209, USA, ATCC accession number VR-2547.

Another useful poxvirus vector is TROVAC. TROVAC refers to an attenuated fowlpox that was a plaque-cloned isolate derived from the FP-1 vaccine strain of fowlpoxvirus which is licensed for vaccination of 1 day old chicks. TROVAC was likewise deposited under the terms of the Budapest Treaty with the ATCC, accession number 2553.

“Non-viral” plasmid vectors may also be suitable in certain embodiments. Preferred plasmid vectors are compatible with bacterial, insect, and/or mammalian host cells. Such vectors include, for example, PCR-II, pCR3, and pcDNA3.1 (Invitrogen, San Diego, Calif.), pBSII (Stratagene, La Jolla, Calif.), pET15 (Novagen, Madison, Wis.), pGEX (Pharmacia Biotech, Piscataway, N.J.), pEGFP-N2 (Clontech, Palo Alto, Calif.), pETL (BlueBacII, Invitrogen), pDSR-alpha (PCT pub. No. WO 90/14363) and pFastBacDual (Gibco-BRL, Grand Island, N.Y.) as well as Bluescript plasmid derivatives (a high copy number COLE1-based phagemid, Stratagene Cloning Systems, La Jolla, Calif.), PCR cloning plasmids designed for cloning Taq-amplified PCR products (e.g., TOPO™ TA cloning® kit, PCR2.1° plasmid derivatives, Invitrogen, Carlsbad, Calif.). Bacterial vectors may also be used with the current invention. These vectors include, for example, Shigella, Salmonella, Vibrio cholerae, Lactobacillus, Bacille calmette guérin (BCG), and Streptococcus (see for example, WO 88/6626; WO 90/0594; WO 91/13157; WO 92/1796; and WO 92/21376). Many other non-viral plasmid expression vectors and systems are known in the art and could be used with the current invention.

Other delivery techniques may also suffice in practicing the present invention including, for example, DNA-ligand complexes, adenovirus-ligand-DNA complexes, direct injection of DNA, CaPO₄ precipitation, gene gun techniques, electroporation, and colloidal dispersion systems. Colloidal dispersion systems include macromolecule complexes, nanocapsules, microspheres, beads, and lipid-based systems including oil-in-water emulsions, micelles, mixed micelles, and liposomes. The preferred colloidal system of this invention is a liposome, which are artificial membrane vesicles useful as delivery vehicles in vitro and in vivo. RNA, DNA and intact virions can be encapsulated within the aqueous interior and be delivered to cells in a biologically active form (Fraley, R., et al., 1981, Trends Biochem. Sci., 6: 77). The composition of the liposome is usually a combination of phospholipids, particularly high-phase-transition-temperature phospholipids, usually in combination with steroids, especially cholesterol. Other phospholipids or other lipids may also be used. The physical characteristics of liposomes depend on pH, ionic strength, and the presence of divalent cations. Examples of lipids useful in liposome production include phosphatidyl compounds, such as phosphatidylglycerol, phosphatidylcholine, phosphatidylserine, phosphatidylethanolamine, sphingolipids, cerebrosides, and gangliosides. Particularly useful are diacylphosphatidylglycerols, where the lipid moiety contains from 14-18 carbon atoms, particularly from 16-18 carbon atoms, and is saturated. Illustrative phospholipids include egg phosphatidylcholine, dipalmitoylphosphatidylcholine and distearoylphosphatidylcholine.

Administration of a targeted immunogen of the present invention to a host may be accomplished using any of a variety of techniques known to those of skill in the art. A composition(s) comprising a targeted immunogen may be processed in accordance with conventional methods of pharmacy to produce medicinal agents for administration to patients, including humans and other mammals (i.e., to produce a “pharmaceutical composition”). The pharmaceutical composition is preferably made in the form of a dosage unit containing a given amount of DNA, viral vector particles, polypeptide or peptide, for example. A suitable daily dose for a human or other mammal may vary widely depending on the condition of the patient and other factors, but, once again, can be determined using routine methods.

The pharmaceutical composition may be administered orally, parentally, by inhalation spray, rectally, or topically in dosage unit formulations containing conventional pharmaceutically acceptable carriers, adjuvants, and vehicles. The term “pharmaceutically acceptable carrier” or “physiologically acceptable carrier” as used herein refers to one or more formulation materials suitable for accomplishing or enhancing the delivery of a nucleic acid, polypeptide, or peptide as a pharmaceutical composition. A “pharmaceutical composition” is a composition comprising a therapeutically effective amount of a nucleic acid or polypeptide. The terms “effective amount” and “therapeutically effective amount” each refer to the amount of a nucleic acid or polypeptide used to induce or enhance an effective immune response. It is preferred that compositions of the present invention provide for the induction or enhancement of an anti-tumor immune response in a host which protects the host from the development of a tumor and/or allows the host to eliminate an existing tumor from the body.

For oral administration, the pharmaceutical composition may be of any of several forms including, for example, a capsule, a tablet, a suspension, or liquid, among others. Liquids may be administered by injection as a composition with suitable carriers including saline, dextrose, or water. The term parenteral as used herein includes subcutaneous, intravenous, intramuscular, intrastemal, infusion, or intraperitoneal administration. Suppositories for rectal administration of the drug can be prepared by mixing the drug with a suitable non-irritating excipient such as cocoa butter and polyethylene glycols that are solid at ordinary temperatures but liquid at the rectal temperature.

The dosage regimen for immunizing a host or otherwise treating a disorder or a disease with a composition of this invention is based on a variety of factors, including the type of disease, the age, weight, sex, medical condition of the patient, the severity of the condition, the route of administration, and the particular compound employed. Thus, the dosage regimen may vary widely, but can be determined routinely using standard methods.

While the compositions of the invention can be administered as the sole active pharmaceutical agent, they can also be used in combination with one or more other compositions or agents. When administered as a combination, the individual components can be formulated as separate compositions administered at the same time or different times, or the components can be combined as a single composition.

A kit comprising a composition of the present invention is also provided. The kit can include a separate container containing a suitable carrier, diluent or excipient. The kit can also include an additional anti-cancer, anti-tumor or antineoplastic agent and/or an agent which reduces or alleviates ill effects of antineoplastic, anti-tumor or anti-cancer agents for co- or sequential-administration. Additionally, the kit can include instructions for mixing or combining ingredients and/or administration.

A better understanding of the present invention and of its many advantages will be had from the following examples, given by way of illustration.

### EXAMPLES

### Example 1

### Preparation of Immunogenic Target Peptides

All peptides were synthesized by Bio-Synthesis Incorporated (Lewisville, Tex.) using standard techniques.

To demonstrate the feasibility of the epitope conjugation system, cytotoxic T lymphocyte (CTL) epitopes were conjugated to the various transduction sequences. The following transcytosis peptides were selected for linking to the epitopes:

| | |
|---|---|
| TAT: | GYGRKKRRQRRR |
| hPER1-1: | SRRHHCRSKAKRSRHH |
| hPER1-2: | GRRHHRRSKAKRSR |
| AntPHD: | RQIKIWFQNRRMKWKK |

Certain of the epitope peptides were joined to the transcytosis sequence using a linker sequence. The linker was selected from the sequence naturally found directly N-terminal to the epitope sequence, or selected based on known immunological parameters. The selected linker sequences are shown below:

| | |
|---|---|
| OVA: | LEQLE (natural) |
| | DEVWEL (synthetic) |
| NP 366-374: | RGVQI |
| gp100 (154-162): | FVYVW |

Several epitopes were selected, as shown below:

| | |
|---|---|
| OVA: | SIINFEKL |
| NP 366-374: | ASNENMETM |
| | (Rotzschke et al. 1990 |
| | Nature 34 8:252) |
| gp100 (280-288(9V)): | YLEPGPVTV |
| | (Parkhurst et al. 1996 |
| | J. Immunol. 257:2539) |
| gp100 (154-162): | KTWGQYWQV |
| | Kawakami et al. 1995. J. |
| | Immunol. 154:3961 |

Several immunogenic targets were then synthesized by combining the above-described transcytosis peptides, linker sequences and epitope peptides, as shown below:

TAT-OVA PEPTIDES:
GYGRKKRRQRRR-SIINFEKL
GYGRKKRRQRRR-LEQLE-SIINFEKL
GYGRKKRRQRRR-DEVWEL-SIINFEKL
hPER1-OVA PEPTIDES:
GRRHHRRSKAKRSRSIINFEKL
GRRHHRRSKAKRSR-LEQL-SIINFEKL
GRRHHRRSKAKRSR-SGQL-SIINFEKL
hPER1-NP PEPTIDES
SRRHHCRSKAKRSRHH-ASNENMETM
GRRHHRRSKAKRSR-ASNENMETM
GRRHHRRSKAKRSR-RGVQI-ASNENMETM
hPER1-1-gp100 (280-288)
SRRHHCRSKAKRSRHH-YLEPGPVTV
hPER1-2-gp100 (154-162)
GRRHHRRSKAKRSR-TWGQYWQV
GRRHHRRSKAKRSR-FVYVW-TWGQYWQV
AntPHD-gp100
RQIKIWFQNRRMKWKK-TWGQYWQV
RQIKIWFQNRRMKKWKK-FVYVW-TWGQYWQV

These peptides were then tested in immunological assays, as described below.

### EXAMPLE 2

### Immunological Testing

A. hPER1-CTL Epitope Conjugates can Form CTL Target Structures When Incubated With Cells In Vitro.

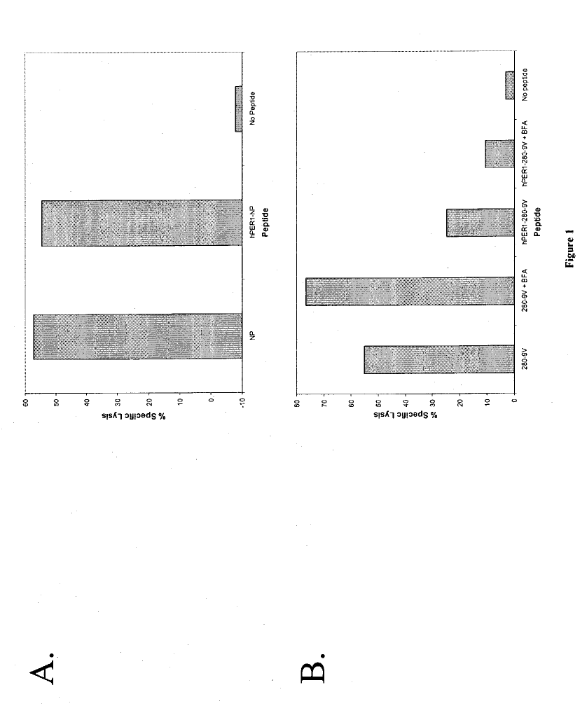

To determine whether hPER1-CTL conjugates can form CTL target structures, ⁵¹Cr-labeled RMA cells were pulsed with 10-11 g/ml NP peptide (ASNENMETM) or hPER1-NP peptide (GRRHHRRSKAKRSRASNENMETM), or were left untreated (no peptide) and incubated for 1 hour at 37° C. The cells were then washed and tested for CTL recognition in a standard 4-hour chromium release assay, using T cells obtained from the spleens of C57BL/6 mice immunized with influenza virus. FIG. 1A demonstrates that RMA target cells can be sensitized for CTL-mediated lysis when incubated with 10 pg/ml of hPERl -NP peptide.

Further, ⁵¹Cr-labeled P815-A2/K^{b} cells were pulsed with 10⁻⁶ g/ml 280-9V peptide (YLEPGPVTV) or hPER1-280-9V (GRRHHRRSKAKRSRYLEPGPVTV) or were left untreated (no peptide) and incubated for 1 hour at 37° C. The cells were then washed and tested for CTL recognition in a standard 4-hour chromium release assay, using T cells obtained from the spleens of HLA-A2/Kb transgenic mice immunized with 280-9V peptide in incomplete Freund's adjuvant. Where indicated, 5 μg/ml brefeldin A (BFA) was included in the assay, to block the surface expression of nascent class I MHC molecules. FIG. 1B demonstrates that P815-A2/K^{b} target cells can be sensitized with 10⁻⁶ g/ml of hPER1-280-9V peptide. The level of CTL killing is reduced if the hPER1-280-9V-pulsed target cells are treated with brefeldin A, which blocks the intracellular transport of newly synthesized MHC molecules.

These experiments demonstrate that hPER1-mediated intracellular delivery provides for increased sensitization of murine T cells. As such, experiments were performed to confirm this effect in human CTL.

B. hPER1-CTL epitope conjugates are immunogenic in a human T cell culture system.

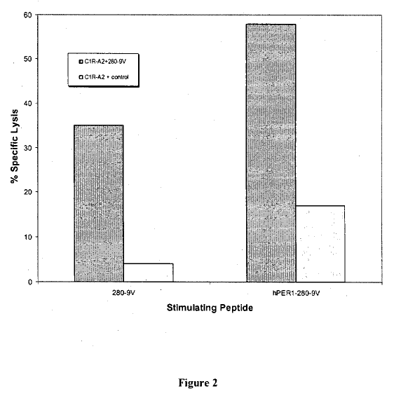

Peripheral blood mononuclear cells (PBMCs) from an HLA-A2-positive patient were cultured in the presence of IL-2 (50 U/ml), IL-7 (10 ng/ml), LPS (10 μg/ml), CD40-ligand expressing 3T3 cells, and peptide (10 μg/ml of 280-9V or hPER1-280-9V). On days 11, 22, and 32 the cells were restimulated by culturing in the presence of IL-2 (50 U/ml) and IL-7 (10 ng/ml) and autologous, CD40-ligand activated PBMCs pulsed with peptide (100 μg/ml of 280-9V or hPER1-280-9V) for 3 hours. On day 42, the cultures were tested for CTL activity in a standard chromium release assay, using C1R-A2 target cells pulsed with 280-9V peptide or a control A2-binding peptide. FIG. 2 demonstrates that 280-9V-specific human CTLs can be induced by repeated in vitro stimulation with hPER1-280-9V.

C. hPER1-CTL Epitope Conjugates are Immunogenic In Vivo, in the Absence of Adjuvant

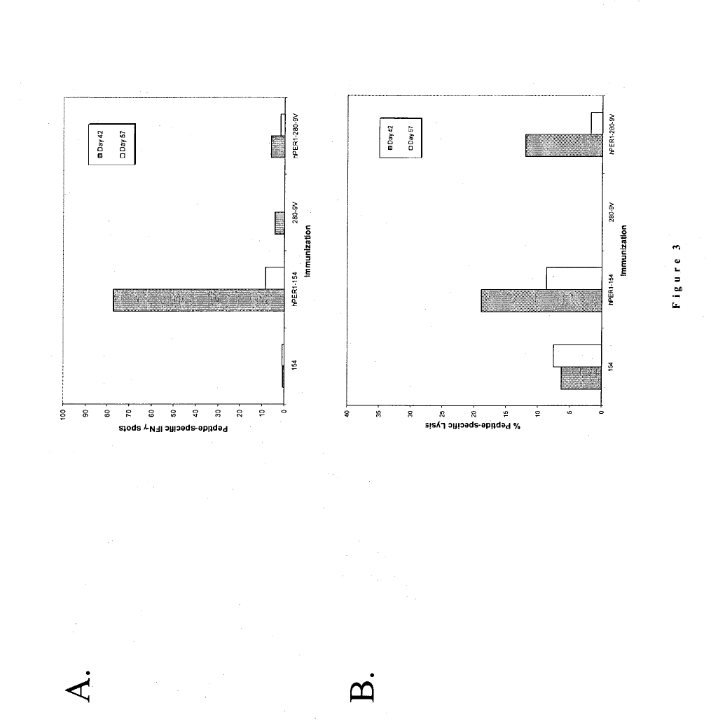

HLA-A2/K^{b} transgenic mice (four per group) were immunized subcutaneously with 100 μg of 154, hPER1-154, 280-9V, or hPER1-280-9V in the presence of an I-A^{b}-restricted T helper epitope (100 μg). Mice were similarly boosted on days 14 and 28. On day 42, splenocytes (2 mice per group) were individually restimulated in vitro for 6 days with the appropriate wild type peptide, and then tested for either IFN-γ secretion by ELISPOT (FIG. 3A) or CTL assay (FIG. 3B) using peptide-pulsed C1R-A2 cells. On day 57, the remaining mice in each group were similarly tested. Average responses from each group are shown.

FIG. 3A demonstrates that 154-specific IFN-γ responses can be induced by immunizing HLA-A2/K^{b} transgenic mice with hPER1-154 (plus a T-helper peptide) in the absence of adjuvant. Similar immunization using the wild type parental peptide fails to induce a response. As shown in FIG. 3B, peptide-specific CTL responses can be induced by immunization with hPER1-154 or hPER1-280-9V, while no responses are induced following immunization with the wild type parental peptides.

D. hPER1-Epitope Conjugation Prolongs the Immune Response

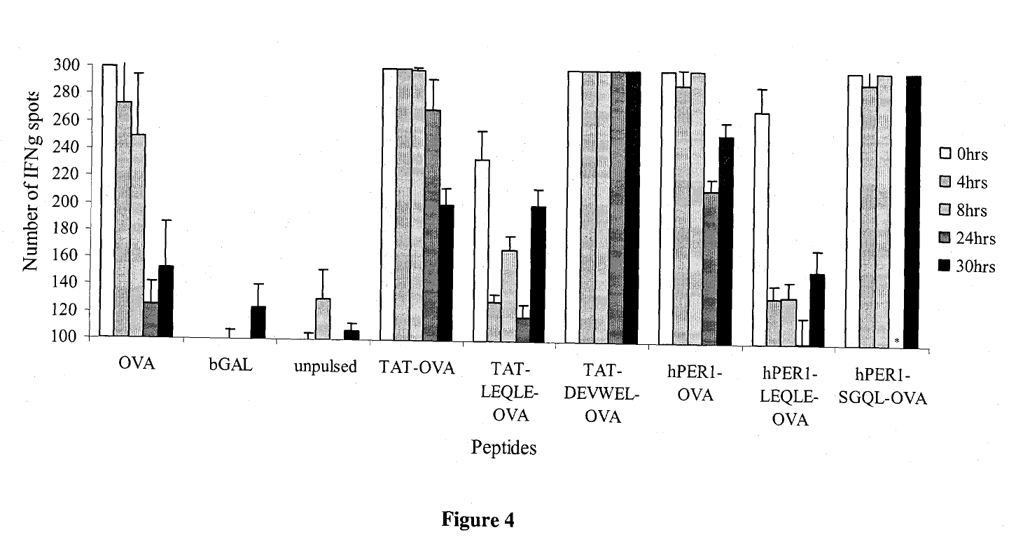

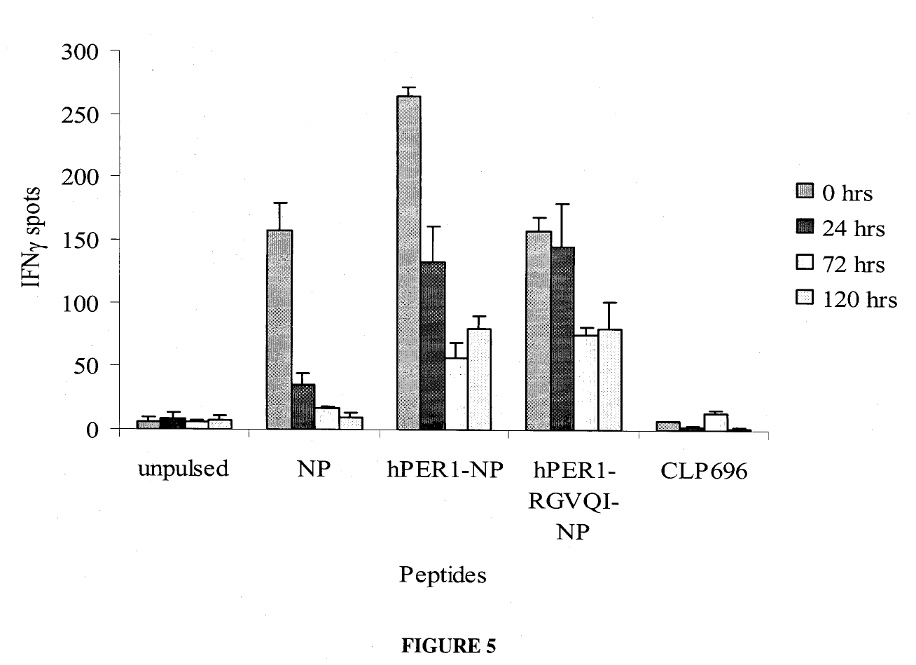

To further study the effect of coupling CTL epitopes to the hPER1 transduction domain, the following in vitro assay was developed to assess the kinetics of antigen presentation following incubation of cells with peptide. In FIG. 4, splenocytes from C57BL/6 mice were incubated with different OVA-based peptides for 1 hour at 37C. The cells were then washed to remove any residual free peptide, and incubated in culture medium at 37C for 0, 4, 8, 24 or 30 hours. The cells were then tested for their ability to stimulate IFN-γ production from SIINFEKL-specific T cells by ELISPOT. The results show that cells pulsed with native OVA peptide lose their stimulatory capacity by 24 hours, whereas cells pulsed with hPER1-SGQL-OVA or TAT-DEVWEL-OVA show no reduction in activity even after 30 hours. Conjugation of OVA to hPER1 or TAT in the absence of linker sequences also enhanced antigen presentation relative to the native OVA peptide, although their activity was lower than the peptides containing the custom designed linkers. hPER1 and TAT conjugates incorporating the natural OVA flanking sequence (LEQLE) as linkers showed no improvement over native peptide. FIG. 5 illustrates a similar analysis performed using the NP system. Here, the native NP peptide shows a loss in activity after 24 hours of incubation. Cells pulsed with the hPER1-NP or hPER1-RGVQI-NP peptide, however, retain their ability to stimulate T cells out to five days, which is the limit of the assay. Overall, these data demonstrate that hPER1 can prolong the duration of antigen presentation, and can be further optimized by the design of an appropriate linker.

E. In Vivo Immunogenicity

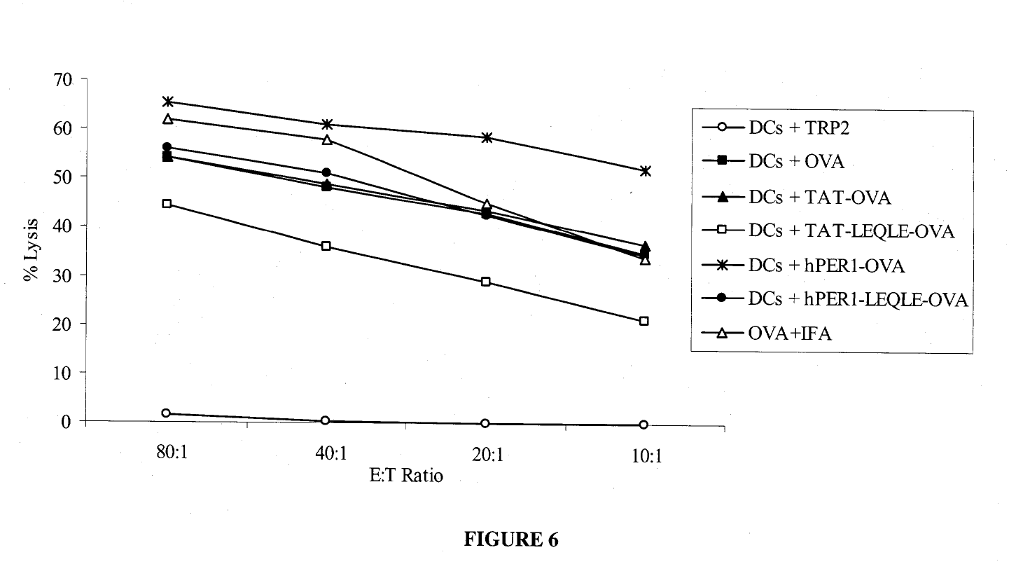

Mature dendritic cells (DCs) are efficient antigen presenting cells that have been shown to generate potent CTL responses following intravenous injection in mice. Consequently, we tested the ability of transcytosis peptides to generate CTL responses in the context of a DC-based vaccine. Murine bone marrow derived dendritic cells were matured in vitro, pulsed with either OVA alone, conjugated with either Tat or hPER1 with or without linkers, and were injected intravenously in the tail vein of C57BL/6 mice. One week post immunization, the splenocytes from vaccinated animals were tested for CTL activity following in vitro restimulation. As shown in FIG. 6, all OVA-pulsed DCs were able to generate potent CTL responses, whereas DCs pulsed with an irrelevant peptide (TRP2) were non immunogenic. DCs pulsed with hPER1-OVA generated a stronger response than either DCs pulsed with native OVA peptide or hPER1-LEQLE-OVA. Similarly, the TAT-LEQLE-OVA peptide was more immunogenic than TAT-OVA without linker, which is consistent with the in vitro observations described above.

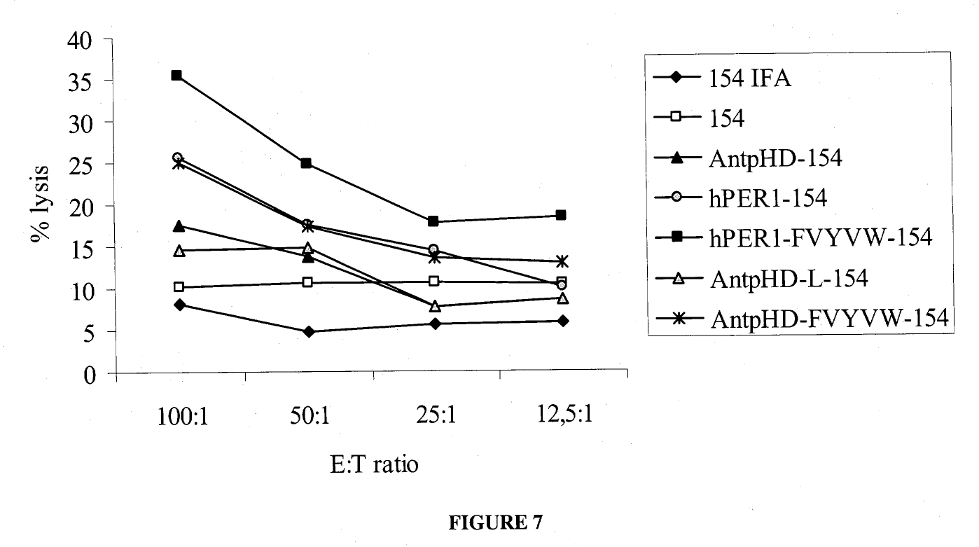

Furthermore, CTL responses were assessed in HLA-A2/K^{b} transgenic mice (Sherman strain) following s.c. immunization with 154 peptide alone, conjugated to hPER1 or AntpHD with or without linker FVYVW. Mice were boosted on days 21 and 42 and splenocytes from vaccinated animals were harvested on day 63 and tested for CTL activity after 5 days of restimulation in vitro. As shown in FIG. 7, 154 peptide alone was unable to generate potent CTL responses even in the presence of incomplete Freund's adjuvant. When associated to AntpHD-154 or hPER1-154 a weak response was observed which increased with the presence of the linker sequence FVYVW. However the most potent activity was observed when the epitope was conjugated to hPER1.

While the present invention has been described in terms of the preferred embodiments, it is understood that variations and modifications will occur to those skilled in the art. Therefore, it is intended that the appended claims cover all such equivalent variations that come within the scope of the invention as claimed.

What is claimed is:

1. A polypeptide consisting essentially of a first amino acid sequence comprising a comprising a transduction sequence of hPER1 linked to a second amino acid sequence comprising a cytotoxic T lymphocyte epitope.

2. The polypeptide of claim 1 wherein a linker sequence is inserted between the first and second amino acid sequences.

3. The polypeptide of claim 2 wherein the linker sequence naturally occurs with the second amino acid sequence.

4. The polypeptide of claim 2 wherein the linker sequence does not naturally occur with the second amino acid sequence.

5. The polypeptide of claim 1 wherein the second amino acid sequence is derived from a tumor antigen.

6. The polypeptide of claim 5 wherein the tumor antigen is a human melanoma antigen.

7. The polypeptide of claim 6 wherein the tumor antigen is gp100, MART-1, tyrosinase, MAGE or TRP2.

8. The polypeptide of claim 5 wherein the second amino acid sequence is selected from the group consisting of YLEPGPVTV (SEQ ID NO: 5); KTWGQYWQV (SEQ ID NO:6); ILTVILGVL (SEQ. ID. NO. 7); GILTVILGV (SEQ. ID. NO.8); NAPPAYEKL (SEQ. ID. NO.9); MPREDAHFI (SEQ. ID.NO.10); ALMDKSLHV (SEQ ID.NO.11); VLLLIGCWY (SEQ. ID. NO. 12); VILGVLLLI (SEQ. ID. NO.13); SLHVGTQCA (SEQ. ID. NO.14); LMDKSLHVG (SEQ. ID.NO.15); ELVHFLLLK (SEQ ID NO: 16); KVLHHMVKI (SEQ ID NO: 17); RALVETSYV (SEQ ID NO: 18); FQAALSRKV (SEQ ID NO: 19); GPHISYPPL (SEQ ID NO: 20); KKLLTQHFV (SEQ ID NO.21); GLVGAQAPA (SEQ ID NO.22); YPPLHEWVL (SEQ ID NO.23); LPTTMNYPL (SEQ ID NO.24); NIYDLFVWM (SEQ ID NO: 25); DLGYDYSYL (SEQ ID NO: 26); NILLSNAPL (SEQ ID NO: 27); SLPYWNFAT (SEQ ID NO: 28); TLGTLCNST (SEQ ID NO: 29); IAVVGALLL (SEQ ID NO: 30); NISIYNYFV (SEQ ID NO: 31); and, NMVPFWPPV (SEQ ID NO: 32).

9. The polypeptide of claim 8 wherein the second amino acid sequence is YLEPGPVTV (SEQ ID NO: 5) or KTWGQYWQV (SEQ ID NO:6).

10. A polypeptide of 1 wherein the first amino acid sequence is SRRHHCRSKAKRSRHH or GRRHHRRSKAKRSR.

11. A polypeptide of claim 7 wherein the first amino acid sequence is SRRHHCRSKAKRSRHH or GRRHHRRSKAKRSR.

12. A polypeptide of claim 8 wherein the first amino acid sequence is SRRHHCRSKAKRSRHH or GRRHHRRSKAKRSR.

13. A polypeptide of claim 9 wherein the first amino acid sequence is SRRHHCRSKAKRSRHH or GRRHHRRSKAKRSR.

11. A composition comprising a polypeptide of claim 1 in a pharmaceutically acceptable carrier.

12. A composition comprising a polypeptide of claim 7 in a pharmaceutically acceptable carrier.

13. A composition comprising a polypeptide of claim 8 in a pharmaceutically acceptable carrier.

14. A composition comprising a polypeptide of claim 9 in a pharmaceutically acceptable carrier.

15. A composition comprising a polypeptide of claim 10 in a pharmaceutically acceptable carrier.

16. A composition comprising a polypeptide of claim 11 in a pharmaceutically. acceptable carrier.

17. A composition comprising a polypeptide of claim 12 in a pharmaceutically acceptable carrier.

18. A composition comprising a polypeptide of claim 13 in a pharmaceutically acceptable carrier.

19. A method for immunizing a host comprising administering to the host a composition of claim 11.

20. A method for immunizing a host comprising admixing a polypeptide or composition of 1 with dendritic cells to generate peptide-loaded dendritic cells and administering the peptide-loaded dendritic cells to the host.

21. An isolated recombinant DNA molecule comprising a first DNA sequence encoding a cytotoxic T lymphocyte epitope and a second DNA sequence encoding a transduction sequence of hPER1.

22. The DNA molecule of claim 21 wherein a DNA sequence encoding a linker amino acid sequence is inserted between the first and second amino acid sequences.

23. The DNA molecule of claim 22 wherein the linker amino acid sequence naturally occurs with the second amino acid sequence.

24. The DNA molecule of claim 23 wherein the linker sequence does not naturally occur with the second amino acid sequence.

25. The DNA molecule of claim 21 wherein the first amino acid sequence is derived from a tumor antigen.

26. The DNA molecule of claim 25 wherein the tumor antigen is a human melanoma antigen.

27. The DNA molecule of claim 26 wherein the tumor antigen is gp100, MART-1, tyrosinase, MAGE or TRP2.

28. The DNA molecule of claim 27 wherein the DNA sequence encoding the second amino acid sequence is selected from the group consisting of

| | |
|---|---|
| TACCTGGAGCCCGGCCCCGTGACCGTG; | (SEQ ID NO.:37) |
| AAGACCTGGGGCCAGTACTGGCAGGTG; | (SEQ ID NO.:38) |
| ATCCTGACAGTGATCCTGGGAGTCTTA; | (SEQ ID NO:39) |
| GGCATCCTGACAGTGATCCTGGGAGTC; | (SEQ ID NO:40) |
| AATGCTCCACCTGCTTATGAGAAACTC; | (SEQ ID NO:42) |
| ATGCCAAGAGAAGATGCTCACTTCATC; | (SEQ ID NO:43) |
| GCCTTGATGGATAAAAGTCTTCATGTT; | (SEQ ID NO:44) |
| GTCTTACTGCTCATCGGCTGTTGGTAT; | (SEQ ID NO:45) |
| GTGATCCTGGGAGTCTTACTGCTCATC; | (SEQ ID NO:46) |
| AGTCTTCATGTTGGCACTCAATGTGCC; | (SEQ ID NO:47) |
| TTGATGGATAAAAGTCTTCATGTTGGC; | (SEQ ID NO:48) |
| GAGTTGGTTCATTTTCTGCTCCTCAAG; | (SEQ ID NO.49) |
| AAAGTCCTGCACCATATGGTAAAGATC; | (SEQ.ID.NO.50) |
| AGGGCCCTCGTTGAAACCAGCTATGTG; | (SEQ ID.NO.51) |
| TTCCAAGCAGCACTCAGTAGGAAGGTG; | (SEQ ID.NO.52) |
| GGACCTCACATTTCCTACCCACCCCTG; | (SEQ.ID.NO.53) |
| AAGAAGCTGCTCACCCAACATTTCGTG; | (SEQ ID.NO.54) |
| GGCCTGGTGGGTGCGCAGGCTCCTGCT; | (SEQ ID NO:55) |
| TACCCACCCCTGCATGAGTGGGTTTTG; | (SEQ ID.NO.56) |
| CTCCCCACTACCATGAACTACCCTCTC; | (SEQ.ID.NO.57) |
| AATATTTATGACCTCTTTGTCTGGATG; | (SEQ ID NO:58) |
| GATCTGGGCTATGACTATAGCTATCTA; | (SEQ ID NO:59) |
| AATATCCTTCTGTCCAATGCACCACTT; | (SEQ ID NO:60) |
| TCCCTTCCTTACTGGAATTTTGCAACG; | (SEQ ID NO:61) |
| ACCCTGGGAACACTTTGTAACAGCACC; | (SEQ ID NO:62) |
| ATAGCAGTAGTTGGCGCTTTGTTACTG; | (SEQ ID NO:63) |
| AACATTTCCATTTATAACTACTTTGTT; | (SEQ ID NO:64) |
| and, | |
| AACATGGTGCCATTCTGGCCCCCAGTC. | (SEQ ID NO:65) |

29. The DNA molecule of claim 28 wherein the DNA sequence encoding the second amino acid sequence is

| | |
|---|---|
| TACCTGGAGCCCGGCCCCGTGACCGTG; | (SEQ ID NO.:37) |
| or | |
| AAGACCTGGGGCCAGTACTGGCAGGTG. | (SEQ ID NO.:38) |

30. The DNA molecule of claim 21 wherein the DNA sequence encoding the first amino acid sequence is
(SEQ ID NO.:35)
AGCAGGAGGCACCACTGCAGGAGCAAGGCCAAGAGGAGCAGGCACCAC;
or
(SEQ ID NO.:36)
GGCAGGAGGCACCACAGGAGGAGCAAGGCCAAGAGGAGCAGG

31. The DNA molecule of claim 27 wherein the DNA sequence encoding the first amino acid sequence is
(SEQ ID NO.:35)
AGCAGGAGGCACCACTGCAGGAGCAAGGCCAAGAGGAGCAGGCACCAC;
or
(SEQ ID NO.:36)
GGCAGGAGGCACCACAGGAGGAGCAAGGCCAAGAGGAGCAGG.

32. The DNA molecule of claim 28 wherein the DNA sequence encoding the first amino acid sequence is
(SEQ ID NO.:35)
AGCAGGAGGCACCACTGCAGGAGCAAGGCCAAGAGGAGCAGGCACCAC;
or
(SEQ ID NO.:36)
GGCAGGAGGCACCACAGGAGGAGCAAGGCCAAGAGGAGCAGG.

33. The DNA molecule of claim 29 wherein the DNA sequence encoding the first amino acid sequence is
(SEQ ID NO.:35)
AGCAGGAGGCACCACTGCAGGAGCAAGGCCAAGAGGAGCAGGCACCAC;
or
(SEQ ID NO.:36)
GGCAGGAGGCACCACAGGAGGAGCAAGGCCAAGAGGAGCAGG.

34. A composition comprising a polypeptide of claim 21 in a pharmaceutically acceptable carrier.

35. A composition comprising a polypeptide of claim 27 in a pharmaceutically acceptable carrier.

36. A composition comprising a polypeptide of claim 28 in a pharmaceutically acceptable carrier.

37. A composition comprising a polypeptide of claim 29 in a pharmaceutically acceptable carrier.

38. A composition comprising a polypeptide of claim 30 in a pharmaceutically acceptable carrier.

39. A composition comprising a polypeptide of claim 31 in a pharmaceutically acceptable carrier.

40. A composition comprising a polypeptide of claim 32 in a pharmaceutically acceptable carrier.

42. A composition comprising a polypeptide of claim 33 in a pharmaceutically acceptable carrier.

43. A method for immunizing a host comprising administering to the host a composition of claim 21.
